(19) United States
(12) Patent Application Publication
(10) Pub. No.: US 2024/0285971 A1
(43) Pub. Date: Aug. 29, 2024

(54) Intratumoral Alpha-Emitter Radiation in Combination with Vasculature Inhibitors

(71) Applicant: ALPHA TAU MEDICAL LTD., Jerusalem (IL)

(72) Inventors: Yona Keisari, Ramat Gan (IL); Itzhak Kelson, Tel Aviv (IL); Vered Domankevich, Hod Hasharon (IL); Yossi Nishri, Jerusalem (IL); Tomer Cooks, Gedera (IL); Margalit Efrati, Netanya (IL); Ronen Segal, Jerusalem (IL); Robert Den, Merion Station, PA (US); Ishai Luz, Kibbutz Gevim (IL); Maayan Hedva Vatarescu, Lehavim (IL); Sara Del Mare Roumani, Modiin Maccabim Reut (IL); Amit Shai, Tel Aviv-Yafo (IL)

(21) Appl. No.: 18/569,636

(22) PCT Filed: Jun. 19, 2022
(86) PCT No.: PCT/IB2022/055679

§ 371 Date: Dec. 13, 2023

Related U.S. Application Data
(60) Provisional application No. 63/212,670, filed on Jun. 20, 2021.

(51) Int. Cl. A61N 5/10; A61K 9/00; A61K 31/4412; A61K 39/00; A61K 51/12; C07K 16/22

(52) U.S. Cl. CPC A61N 5/1001; A61K 9/0019; A61K 31/4412; A61K 51/1282; C07K 16/22; A61K 2039/505; A61K 2039/545; A61N 2005/101; A61N 2005/1024

(57) ABSTRACT

A vascular inhibitor for use as a medicament for treatment of a tumor of a patient wherein the administration pattern of the medicament comprises administering a therapeutically effective amount of the vascular inhibitor to the patient, in one or more sessions, and implanting seeds (204) carrying radium-224 in the tumor for intra-tumoral alpha-emitter radiotherapy less than two weeks from administering the vascular inhibitor.

### FIELD OF THE INVENTION

The present invention relates generally to tumor therapy and particularly to combined intra-tumoral alpha-emitter radiation and vascular inhibitors.

### BACKGROUND OF THE INVENTION

Cancer is the primary cause of death in many countries around the world. Accordingly, an enormous volume of resources has been spent on treatments for cancer, and a wide variety of such treatments have been suggested.

One class of tumor therapy is tumor ablation, to kill tumor cells in situ. Multiple ablation methods have been proposed, such as, heat, microwave, laser, electric, photodynamic, chemical (e.g., using reactive oxygen species (ROS)) and radioactive ablation, which could be applied externally (e.g., external beam radiation therapy) or internally (e.g., brachytherapy), and can include different types of radiation such as alpha radiation, beta radiation and gamma radiation. A discussion of these methods appears, for example, in Keisari, Yona. “Tumor abolition and antitumor immunostimulation by physico-chemical tumor ablation.” Front Biosci 22 (2017): 310-347. The ablation method used for any specific patient is generally selected according to the type of the tumor, its location, its stage and/or other parameters of the tumor.

Many other cancer therapy modalities have been employed, such as: surgery. chemotherapy, immunotherapy, DNA repair inhibitors, targeted therapy, hormone treatments, antiangiogenic therapy, and epigenetic modulation. Generally, the specific method used for each patient is selected according to the type of the tumor, location or its stage. Multiple combinations of the above discussed therapy types were tested in pre-clinical and clinical trials. Several of these methods involve the modulation of tumor vasculature, such as antiangiogenic therapy, which involves the inhibition of new blood vessels growth in a patient.

Ionizing radiation destroys cells by creating damage to their DNA. The biological effectiveness of different types of radiation in killing cells is determined by the type and severity of the DNA lesions they create. Alpha particles are a powerful means for radiotherapy since they induce clustered double-strand breaks on the DNA, which cells cannot repair. Unlike conventional types of radiation, the destructive effect of alpha particles is also largely unaffected by low cellular oxygen levels, making them equally effective against hypoxic cells, whose presence in tumors is a leading cause of failure in conventional radiotherapy based on photons or electrons. In addition, the short range of alpha particles in tissue (less than 100 micrometers) ensures that if the atoms which emit them are confined to the tumor volume, surrounding healthy tissue will be spared. On the other hand, the short range of alpha radiation has so far limited their use in cancer therapy, as there was no practical way to deploy alpha emitting atoms in sufficient concentrations throughout the entire tumor volume

Diffusing alpha-emitters radiation therapy (DaRT), described for example in U.S. Pat. No. 8,834,837 to Kelson, extends the therapeutic range of alpha radiation, by using radium-223 or radium-224 atoms, which generate chains of several radioactive decays with a governing half-life of 3.6 days for radium-224 and 11.4 days for radium-223. In DaRT, the radium atoms are attached to a source (also referred to as a “seed”) implanted in the tumor with sufficient strength such that they do not leave the source in a manner that they go to waste (by being cleared away from the tumor through the blood), but a substantial percentage of their daughter radionuclides (radon-220 in the case of radium-224 and radon-219 in the case of radium-223) leave the source into the tumor, upon radium decay. These radionuclides, and their own radioactive daughter atoms and further daughter radionuclides chaining therefrom, spread around the source by diffusion up to a radial distance of a few millimeters before they decay by alpha emission. Thus, the range of destruction in the tumor is increased relative to radionuclides which remain with their daughters on the source.

US patent publication 2020/0276164 to Waugh et al., titled: “Pharmaceutical Combinations for the Treatment of Cancer”, describes treatment of metastatic prostate cancer with androgen deprivation therapy using the combination of vascular endothelial growth factor (VEGF) signaling inhibitor and an interleukin-8 signaling inhibitor, with radiotherapy.

US patent publication 2020/0093968 to Kaplan, titled: “Flexible and/or Elastic Brachytherapy Seed or Strand” describes a flexible brachytherapy strand which provides a drug with the brachytherapy.

### SUMMARY OF THE INVENTION

An aspect of some embodiments of the invention relates to tumor treatment based on a combination between vascular inhibitors, which destroy blood vessels and/or prevent generation and/or growth of new blood vessels, and intra-tumoral diffusing alpha-emitters radiation therapy (DaRT). The term intra-tumoral refers herein to a treatment in which alpha-emitter radionuclides are implanted on a seed within a tumor in one or more initial locations and they or their daughter radionuclides travel to other locations in the tumor, at which alpha-emitting decay occurs. The travel of the radionuclides from the seed may be due to diffusion or due to decay, when the radionuclides on the seed begin a chain of a plurality of radioactive decays.

There is therefore provided in accordance with embodiments of the present invention, a vascular inhibitor for use as a medicament for treatment of a tumor of a patient wherein the administration pattern of the medicament comprises administering a therapeutically effective amount of the vascular inhibitor to the patient, in one or more sessions, and implanting seeds carrying radium-224 in the tumor for intra-tumoral alpha-emitter radiotherapy less than two weeks from (either before or after) administering the vascular inhibitor.

Optionally, the administration pattern of the medicament comprises beginning the administering of the vascular inhibitor within less than five days from implanting of the seeds. Optionally, the administration pattern of the medicament comprises beginning the administering of the vascular inhibitor at least 12 hours after the implanting of the seeds. Optionally, the administration pattern of the medicament comprises beginning the administering of the vascular inhibitor at least 72 hours after the implanting of the seeds. Optionally, the administration pattern of the medicament comprises beginning the administering of the vascular inhibitor at least 12 hours before the implanting of the seeds. Optionally, the administration pattern of the medicament comprises beginning the administering of the vascular inhibitor at least 72 hours before the implanting of the seeds.

Optionally, the vascular inhibitor comprises an antiangiogenic agent. Optionally the antiangiogenic agent comprises a vascular endothelial growth factor (VEGF) blockade. In some embodiments, the VEGF blockade comprises an antibody against vascular endothelial growth factors (VEGFs). Optionally, the antibody against VEGFs comprises Bevacizumab. Optionally, the VEGF blockade comprises a vascular endothelial growth factor (VEGF) decoy receptor. Optionally, the VEGF decoy receptor comprises Ziv-aflibercept. Optionally, the VEGF blockade comprises an antibody against VEGF receptors. Optionally, the antiangiogenic agent comprises a Kinase inhibitor. Optionally, the Kinase inhibitor comprises at least one of Sorafenib, Sunitinib, Regorafenib and Lenvatinib. Optionally, the antiangiogenic agent comprises an Immunomodulatory imide drug (IMiD). In some embodiments, the Immunomodulatory imide drug (IMiD) comprises at least one of Thalidomide, Pomalidomide and lenalidomide.

Optionally, the antiangiogenic agent comprises an antiangiogenic microRNA, an Endothelin receptor blocker, and/or Bosentan. In some embodiments, the antiangiogenic agent comprises an mTOR inhibitor, rapamycin, a Fibroblast Growth Factors inhibitor and/or Brivanib. Optionally, the antiangiogenic agent comprises an Angiopoietins inhibitor. Optionally, the antiangiogenic agent comprises a Platelet-Derived Growth Factor inhibitor. Optionally, the antiangiogenic agent comprises ponatinib. Optionally, the antiangiogenic agent comprises a Hepatocyte Growth Factor (HGF)/c-MET inhibitor. Optionally, the antiangiogenic agent comprises a naturally anti-angiogenic factor or a derivative or a mimic of a naturally anti-angiogenic factor. Optionally, the antiangiogenic agent comprises Endostatin. Optionally, the antiangiogenic agent comprises one or more β-Adrenergic Drug. Optionally, the antiangiogenic agent comprises Propranolol. Optionally, the antiangiogenic agent comprises an angiogenesis inhibitor. Optionally, the antiangiogenic agent comprises Metformin or Chloroquine. Optionally, the antiangiogenic agent comprises a Cannabinoid. Optionally, the antiangiogenic agent comprises a Matrix metalloproteinase inhibitor. Optionally, the antiangiogenic agent comprises an inhibitor of integrin's pro-angiogenic activity. Optionally, the vascular inhibitor comprises a vascular disrupting agent. Optionally, the vascular disrupting agent comprises Combretastatin A-4 phosphate (CA4P).

Optionally, the source comprises a support having a length of at least 1 millimeter; and radium-224 atoms coupled to the support such that not more than 20% of the radium-224 atoms leave the support into the tumor in 24 hours, without decay, when the source is implanted in the tumor, but upon decay, at least 5% of daughter radionuclides of the radium-224 atoms leave the support upon decay.

There is further provided in accordance with embodiments of the present invention, a method of treating a patient with a tumor, comprising treating the tumor with intra-tumoral alpha-emitter radiotherapy; and administering, to the patient, a vascular inhibitor within two weeks of beginning the treating of the tumor with intra-tumoral alpha-emitter radiotherapy. Optionally, administering the vascular inhibitor comprises administering a vascular endothelial growth factor (VEGF) blockade.

Optionally, treating the tumor with intra-tumoral alpha-emitter radiotherapy comprises treating a colorectal cancer tumor. Optionally, administering the vascular inhibitor comprises administering Bevacizumab and/or Ranibizumab. Optionally, administering the vascular inhibitor comprises administering an antiangiogenic agent. Optionally, administering the vascular inhibitor comprises administering a vascular endothelial growth factor (VEGF) inhibitor. Optionally, administering the vascular inhibitor comprises administering a vascular disrupting agent. Optionally, treating the tumor with intra-tumoral alpha-emitter radiotherapy comprises implanting a plurality of seeds each having a length of at least 1 millimeter, in the tumor. Optionally, administering the vascular inhibitor comprises administering within less than five days from implanting of the seeds. Optionally, administering the vascular inhibitor comprises administering at least 12 hours after the implanting of the seeds.

There is further provided in accordance with embodiments of the present invention, a kit for treatment of a patient, comprising at least one source for being at least partially introduced into a body of a subject, having alpha-emitting atoms mounted thereon, at least one vascular inhibitor; and a package containing the at least one source and the at least one vascular inhibitor.

There is further provided in accordance with embodiments of the present invention, an alpha-emitting device designed for use in alpha-emitter radiotherapy treatment of a tumor of a patient, wherein the alpha-emitter radiotherapy treatment pattern comprises treating the tumor with the alpha-emitter device followed by administering a therapeutically effective amount of a vascular inhibitor, in one or more sessions, less than six weeks after beginning the alpha-emitter radiotherapy.

Optionally, the alpha-emitter radiotherapy treatment pattern comprises treating the tumor with the alpha-emitter device followed by administering a therapeutically effective amount of a vascular inhibitor, in one or more sessions, less than two weeks after beginning the alpha-emitter radiotherapy.

There is further provided in accordance with embodiments of the present invention, an alpha-emitting device designed for use in population treated for a tumor with the alpha-emitter device followed by administering a therapeutically effective amount of a vascular inhibitor, in one or more sessions, less than six weeks after beginning the alpha-emitter radiotherapy.

The various options and alternatives listed in the following description and claims may be used in the alternative or together in any suitable combination, except where the options are specifically contradictory.

### BRIEF DESCRIPTION OF THE DRAWINGS

FIG. 1 is a flowchart of a therapy method, in accordance with an embodiment of the invention;

FIG. 2 is a schematic illustration of a kit for combined alpha-emitter radiation and vascular inhibitors, in accordance with an embodiment of the invention;

FIG. 3 is a graph showing results of an experiment testing an effect of a combined alpha-emitter radiation and Bevacizumab administered after implanting the alpha-emitter radiation seeds, in accordance with an embodiment of the invention;

FIG. 4 is a graph showing results of an experiment testing an effect of a combined alpha-emitter radiation and Bevacizumab which was administered from 4 days before implanting the alpha-emitter radiation seeds, in accordance with an embodiment of the invention; and

FIGS. 5A and 5B show the results of an experiment applicant performed to investigate the effect of the method of FIG. 1 on the effective diameter of DaRT seeds and on the leakage of the radioactive atoms from the tumor.

### DETAILED DESCRIPTION OF EMBODIMENTS

An aspect of some embodiments of the invention relates to a combined tumor treatment including Diffusing alpha-emitters Radiation Therapy (DaRT) and a therapeutically effective treatment of vascular inhibitors. Applicant has found that the combination of vascular inhibitors along with the specific tumor ablation of applying DaRT has a substantially greater therapeutic effect than each of the treatments separately.

The effective range of destruction around DaRT sources carrying the radionuclides is limited to several millimeters and depends on biological attributes of the tumor. Contrary to common wisdom that the chaotic structure of the tumor vasculature increases the spread of DaRT-related radionuclides in the tumor, relative to normal tissue, and therefore the use of vascular inhibitors would reduce the effective range of destruction, applicant found that vascular inhibitors increase the effective range of the DaRT treatment. It is believed by applicant that the increase in the effective range of DaRT resulting from the vascular inhibitors, is due to reduction of blood vessels or prevention of neovascularization, which decrease led leakage from the tumor through the blood stream and thus allow the accumulation of alpha-emitting atoms in the tumor in a manner which increases the effectivity of the alpha-emitter radiation treatment.

It is noted that, at first, applicant thought that the vascular inhibitors should be taken only several days after the beginning of the DaRT treatment, so that the vascular inhibitors do not interfere at the primary period of spread of radionuclides in the tumor. However, after further research, applicant determined that even when vascular inhibitors are administered before beginning the DaRT treatment, their effect is beneficial.

Intra-tumoral alpha-emitter radiation therapy utilizes alpha-emitting atoms for the treatment of tumors by employing a seed (also referred to as “a source”) coated with radioactive atoms. The alpha emitting atoms are released locally, in a controlled-release manner, both in time and in space. Namely, the atoms spread in the tumor gradually by migration and convection. At an initial timepoint most of the radioactivity is concentrated near the source. As time passes, this distribution changes in a way that some of the alpha-emitting atoms in the decay chain of the radionuclides on the source reach more distant locations in the tumor. In addition to the changes of the migration distance with time, in each timepoint there is a different distribution of activity as a function of the distance from the source. Namely, for each given timepoint and migration distance there is a different amount of activity along the line between the source and the migration maximal distance, where shorter distance from the source displays higher activity. This enables a non-unified and non-imrnediate destruction of the tumor tissue. It was shown that the alpha-emitting atoms spread more efficiently in tumor tissue relative to normal tissue, probably due to the abnormal tumor blood vessels structure of the tumor. However, as the radioactive decay chain progresses, together with the dispersion of alpha emitting atoms in the tumor away from the radioactive seed, the chance for the atoms to be evacuated by the blood stream increases. Administering a vasculature inhibitor to the tumor could reduce the leakage of the radionuclides from the tumor.

### Treatment Method

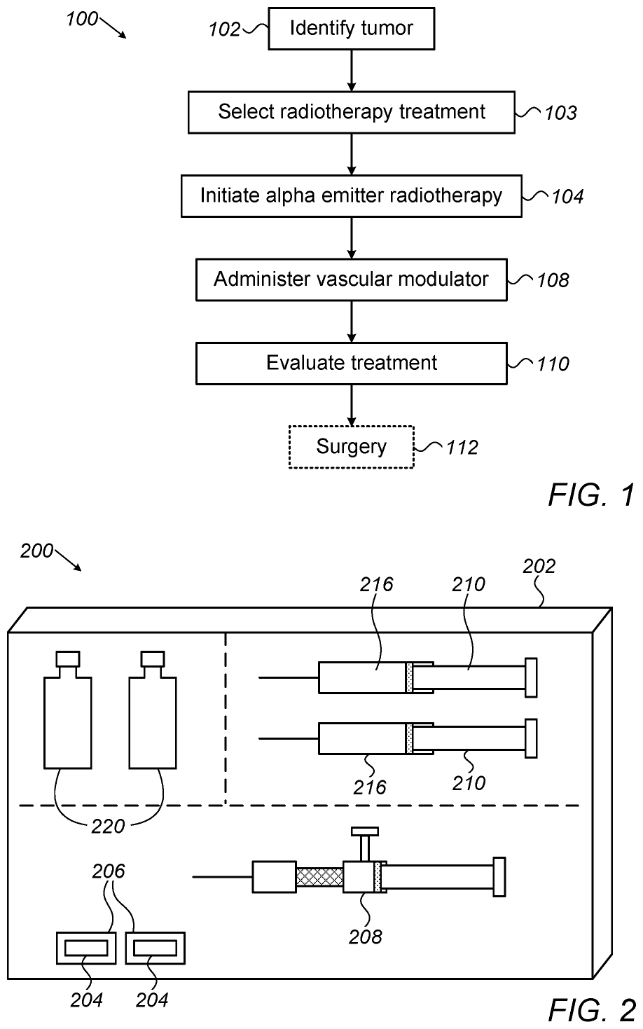

FIG. 1 is a flowchart of a therapy method 100, in accordance with an embodiment of the invention. Further to identification (102) of a tumor in a patient, an alpha-emitter radiation treatment (referred to herein also as alpha-emitter radiotherapy) of the tumor is selected (103) for the tumor. The selection of the treatment optionally includes selecting, responsive to the type of the tumor, an activity of radium-224 on sources to be implanted in the tumor and a spacing between the sources. Thereafter, sources with the selected activity are implanted (104) inside the tumor. In addition, a therapeutically effective dose of one or more vascular inhibitors is administered (108) to the patient in one or more sessions.

In some embodiments, after the alpha-emitter radiation treatment is completed, the effect of the treatment is evaluated (110). In some embodiments, following the evaluation, surgery to remove the residual primary tumor is carried out (112). The surgery is carried out, for example, in patients who were not eligible for surgery before the treatment, following tumor shrinkage due to the treatment. Alternatively or additionally, surgery (112) is carried out before the DaRT treatment.

### Tumor Types

Therapy method 100 may be used in treatment of any tumor type, including cancerous tumors, benign neoplasms, in situ neoplasms (pre-malignant), malignant neoplasms (cancer), and neoplasms of uncertain or unknown behavior. In some embodiments, the method of FIG. 1 is used to treat relatively solid tumor lesions, such as breast, kidney, pancreatic, skin, head and neck, colorectal, ovarian, bladder, brain and prostate cancer. In other embodiments, the therapy method 100 of FIG. 1 is used to treat non-solid tumors. The method of FIG. 1 may be used for both primary and secondary tumors.

Exemplary tumors that can be treated by the method of FIG. 1 include but are not exclusive to tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer; breast cancer, susceptibility to breast cancer, type 4 breast cancer, breast cancer-1, breast cancer-3; breast-ovarian cancer), squamous cell carcinoma (e.g., in head and neck), vulvar cancer, neurogenic tumor, astrocytoma, ganglioblastoma, neuroblastoma, gliomas, adenocarcinoma, adrenal tumor, hereditary adrenocortical carcinoma, brain malignancy (tumor), various other carcinomas (e.g., bronchogenic large cell, ductal, epidermoid, large cell, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, solid lymphoma, liposarcoma, lynch cancer family syndrome II, male germ cell tumor, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

In some embodiments, the method of FIG. 1 is applied to a tumor known to be affected substantially by alpha-emitter radiation on its own. In other embodiments, the method of FIG. 1 is applied to a tumor of a type which is not affected substantially by alpha-emitter radiation on its own, for example does not reduce in size at all or does not reduce in size by more than 5% or 10%. Experiments performed by applicant indicate that even tumors that are not affected substantially by alpha-emitter radiation or vascular inhibitors on their own reduce in size when targeted by the combination of vascular inhibitors and alpha-emitter radiation in accordance with the method of FIG. 1.

While therapy method 100 may be used with any of the above-mentioned tumors, applicant has determined that method 100 is particularly useful for tumors for which the required spacing between the sources is relatively short, e.g., below 4 mm, such as pancreas, melanoma, prostate and glioblastoma, as described in PCT patent application PCT/IB2022/055322, titled “Activity Levels for Diffusing Alpha-emitter Radiation Therapy”, the disclosure of which is incorporated herein by reference in its entirety. Another criterion of tumors which particularly benefit from the method of FIG. 1 are tumors with difficult access, such that the required spacing between the sources is smaller than the spacing which is achievable due to the limited access. For example, the method of FIG. 1 is particularly useful for glioblastoma in the head, as the access to a tumor within a patient's skull is limited and the required spacing is small. Other tumors which are believed to particularly benefit from the method of FIG. 1 are tumors which produce large amounts of a vascular endothelial growth factor (VEGF) and/or have a large amount of blood vessels.

In some embodiments, the decision as to whether to administer (108) the vascular inhibitor is taken responsive to one or more parameters of the radiotherapy treatment. For example, the vascular inhibitor may be administered (108) only in cases in which the selected spacing between the implanted alpha-emitter sources is greater than a predetermined threshold, e.g., 3.6 mm, 3.8 mm or 4 mm. Alternatively, after implantation of the alpha-emitter sources, the actual largest distance between the sources and/or an average distance between the sources is determined, and the vascular inhibitor is administered when the actual distance is substantially larger (for example at least 20%, at least 30% or at least 40%) than the selected spacing. In some embodiments, a minimal radiation dose expected to reach every point in the tumor is calculated for the selected radiotherapy treatment, for example using any of the methods described in PCT application PCT/IB2021/061607, titled: “Treatment Planning for Alpha-Particle Radiotherapy”, the disclosure of which is incorporated herein by reference in its entirety. The calculated radiation dose is compared in these embodiments to an estimated required dose for the tumor type, and the vascular inhibitor is administered only if the calculated radiation dose is not greater than the estimated required dose by a sufficient safety factor.

Alternatively to using the above determination to decide whether to administer (108) the vascular inhibitor, one or more of the above determinations may be used to decide a dose and/or duration of administering the vascular inhibitor. In some embodiments, in accordance with this alternative, the vascular inhibitor is administered before the alpha emitter sources are implanted. After the alpha emitter sources are implanted, the distance between the sources is evaluated and accordingly it is decided whether to continue or terminate the administering of the vascular inhibitor.

In other embodiments, the selection (103) of the parameters of the radiotherapy treatment is performed responsive to whether the patient receives vascular inhibitor treatment with the radiotherapy. Optionally, the activity of the sources, is selected responsive to whether the patient receives vascular inhibitor treatment. For example, for patients with resistance to vascular inhibitors, a higher source activity, e.g., at least 5% higher, at least 10% higher or even at least 20% higher, than used in patients to which vascular inhibitors are administered, is selected. Alternatively or additionally, the spacing between the sources is selected responsive to whether the patient receives vascular inhibitor treatment. Optionally, in accordance with this alternative, for patients with resistance to vascular inhibitors, a shorter spacing, e.g., at least 0.1 millimeter shorter, at least 0.2 millimeters shorter or even at least 0.3 millimeters shorter, than used in patients to which vascular inhibitors are administered, is selected.

### Vascular Inhibitors

In some embodiments, the administered vascular inhibitor comprises an antiangiogenic agent. Optionally the antiangiogenic agent comprises a vascular endothelial growth factor (VEGF) blockade.

Optionally, the VEGF blockade comprises an antibody against VEGF. The VEGF blockade includes, in some embodiments, a monoclonal antibody, such as Bevacizumab (also known under the brand name Avastin), 2C3 and/or Ranibizumab, which target VEGF factors. In other embodiments, the administered VEGF blockade comprises an antibody against VEGF receptors, such as Ramucirumab (also known under the brand name Cyramza), which targets VEGF receptors (VEGFR-2, also known as Kinase insert domain receptor).

Alternatively, or additionally, the administered VEGF blockade comprises a VEGF decoy receptor, such as Ziv-aflibercept (also known as VEGF-trap) and/or Conbercept (sold under the commercial name Lumitin).

In other embodiments, the administered antiangiogenic agent comprises a drug which is not a VEGF blockade, but was identified to have an antiangiogenic effect through a biological pathway other than pathways of VEGF blockades. These antiangiogenic agents which act on other biological pathways are used with DaRT, for example, in patients who are sensitive to VEGF blockades, in patients who do not respond to VEGF blockades, and/or in patients who are already receiving the antiangiogenic agent acting through another pathway for a different purpose than its antiangiogenic properties. In such cases, the antiangiogenic agent may be administered to the patient closer to the time of the DaRT treatment, rather than at an unrelated more distant time, in order to increase its effect and the range of the DaRT treatment.

In accordance with these other embodiments, the antiangiogenic agent optionally comprises a Kinase inhibitor such as Sorafenib (also known as Nexavar), Sunitinib (also known under the brand name Sutent), Regorafenib (also known under the brand name Stivarga) and/or Lenvatinib (also known under the brand name Lenvima). Alternatively, or additionally, the administered Kinase inhibitor comprises Axitinib, Vandetanib, Pazopanib, Cabozantinib, cilengitide, cediranib, enzastaurin and/or Vatalanib.

In still other embodiments, the administered antiangiogenic agent comprises an Immunomodulatory imide drug (IMiD), such as Thalidomide, Pomalidomide, Amiodarone lenalidomide, iberdomide, linomide and/or apremilast. Another option to serve as the administered antiangiogenic agent, in some embodiments, comprises an antiangiogenic microRNA.

It is noted that at least some of these antiangiogenic agents additionally inhibit immune checkpoint expression, and thus increase the effect of the treatment both by preventing regrowth of blood vessels and by increasing the response of the immune system.

Alternatively or additionally, the administered antiangiogenic agent comprises an mTOR inhibitor, such as Everolimus, rapamycin and/or itraconazole. Further alternatively or additionally, the administered antiangiogenic agent comprises a Fibroblast Growth Factors inhibitor, such as Brivanib, dovitinib and/or S49076.

In other embodiments, the administered antiangiogenic agent comprises an Angiopoietins inhibitor, such as vanucizumab, a Platelet-Derived Growth Factor inhibitor, such as ponatinib and/or a Hepatocyte Growth Factor (HGF)/c-MET inhibitor, such as Onartuzumab. The administered antiangiogenic agent includes in other embodiments, a naturally anti-angiogenic factor or its derivative or its mimic, such as thrombospondin (TSP-1 and 2), ATB-510, 3TSR, Pigment epitheliun-derived factor (PEDF), angiostatin and/or Endostatin.

In some embodiments, the administered antiangiogenic agent comprises one or more (3-Adrenergic Drugs, such as carvedilol, nebivolol, Propranolol, metoprolol, bisoprolol, and/or avP3 integrin inhibitors. In other embodiments, the administered antiangiogenic agent comprises a drug that inhibits angiogenesis (also referred to as an angiogenesis inhibitor), such as Metformin, Chloroquine, carboxyamidotriazole, TNP-470, suramin, SU5416, Anecortave acetate, Beloranib, Fluoromedroxyprogesterone acetate, and/or Tasquinimod. In other embodiments, the administered antiangiogenic agent comprises Cannabinoids, such as HU-336, HU-345, Matrix metalloproteinase inhibitors, such as Marimastat, Prinomastat, rebimastat, Neovastat, Batimastat and/or Tanomastat, Inhibitors of integrin's pro-angiogenic activity, such as Medi-522, EMD12194 (Cilengitide), and/or anti-angiogenic gene therapy, such as VB-111.

The administered antiangiogenic agent comprises, in some embodiments, a transforming growth factor-β (TGF-β) inhibitor. In some embodiments, the administered vascular inhibitor includes Endothelin receptor blockers such as Bosentan and/or Atrasentan.

In some embodiments, instead of an antiangiogenic agent, the administered vascular inhibitor comprises a vascular disrupting agent (VDA, also known as vascular targeting agent). While vascular disrupting agents are not known to prevent growth of blood vessels, they are known to disrupt blood vessels, and applicant believes that at least for some tumor types they could be helpful in extending the effective range of DaRT sources. Vascular disrupting agents are used with DaRT, for example in patients who are sensitive to antiangiogenic agents, and/or in patients who are already receiving a vascular disrupting agent for other reasons. In such cases, the vascular disrupting agent may be administered to the patient around the time of the DaRT treatment, in order to increase its effect and the range of the DaRT treatment.

The vascular disrupting agents (VDAs) optionally comprise small molecule and ligand directed VDAs, such as flavonoids or Tubulin-binding agents. The flavonoids may include, for example, Endothelial permeability enhancers, such as Vadimezan (also known as dimethylxanthine acetic acid (DMXAA), 5,6-Dimethylxanthenone-4-acetic acid or ASA404). The Tubulin-binding agents may include Combretastatin A-4 phosphate (CA4P) also known as Zybrestat, Ombrabulin (also known as AVE8062), ZD6126, ABT-571, MN-029 provided by MediciNova, OXi4503, plinabulin (NPI-2358) Combretastatin, AS1404 and/or TZT-1027.

The administered vascular inhibitor may include a single drug, or may include a combination of a plurality of different drugs of the above, administered together or in separate sessions.

### Route of Administration

In some embodiments, the delivery of the vascular inhibitor to the tumor and/or to metastases is done by systemic administration, for example orally or by intravenous (IV) injection or infusion. In some embodiments, the delivery of the vascular inhibitor uses a suitable method of targeted delivery.

Alternatively or additionally, the vascular inhibitor is administered (108) in situ, directly to one or more identified tumors. Optionally, in this alternative the vascular inhibitor is administered by intra-tumoral injection. While in some embodiments the vascular inhibitor is administered from the seeds which carry the alpha-emitter radionuclides, preferably the vascular inhibitor is administered separately from the seeds, in order to achieve a wider coverage of the tumor which receives and is affected by the vascular inhibitor.

Optionally, before administering the vascular inhibitor to a patient, a size of the tumor, tumors and/or metastases is estimated and accordingly an amount of the vascular inhibitor is selected.

### Timing

In some embodiments, the vascular inhibitor is administered (108) in a single session. Alternatively, the vascular inhibitor is administered (108) in multiple sessions, possibly at least three, at least five or even at least seven sessions. The separate sessions are optionally separated by at least four hours, eight hours, 24 hours, 48 hours or even at least 72 hours, from each other. In one specific embodiment, the vascular inhibitor bevacizumab (BEV) is administered over a period of 3 weeks, three times a week. For embodiments in which the vascular inhibitor is administered (108) in multiple sessions, the following paragraphs relate to the first session of administration, unless stated otherwise.

In a first class of embodiments, the vascular inhibitor is administered before the implanting of the alpha-emitting seeds, so that the vascular inhibitor takes effect substantially throughout the duration of the radiotherapy. This class of embodiments is based on experiment results which show that even when the vascular inhibitor is administered before the beginning of the alpha-emitter radiation treatment, e.g., the implanting of the seeds, the combination of DaRT and the vascular inhibitor has a beneficial effect on the destruction of tumor cells. The vascular inhibitor is optionally administered in this class of embodiments, at least 6 hours, at least 12 hours, at least 24 hours or even at least 48 hours, at least 72 hours, at least 96 hours or even at least a week before the beginning of the alpha-emitter radiation treatment. Alternatively or additionally, the vascular inhibitor is administered a short time before the implanting of the alpha-emitting seeds, so that the vascular inhibitor takes effect only after the dispersion of the daughter radionuclides in the tumor begins. For example, the vascular inhibitor is administered in accordance with this alternative less than 72 hours, less than 48 hours, less than 24 hours, less than 12 hours or even less than 6 hours before the implanting of the seeds.

In a second class of embodiments, the timing of the vascular inhibitor therapy is selected so that at first, alpha-emitter radiation is applied without vascular inhibitors, so that radioactive atoms are allowed to exploit the abnormal vascular structure of the tumor for optimized dispersion in the tumor. Thereafter, the vascular inhibitors are added to prevent leakage of atoms from the tumor and reestablishment of residual tumor cells.

Optionally, in this second class of embodiments, the administering of the vascular inhibitor begins a limited buffering time period (106) after implanting the alpha-emitting sources (104). The buffering time period is optionally selected to have the vascular inhibitor take effect, when the radiation activity in the tumor remote from the seed reaches a maximal level. For example, when the diffusing alpha-emitters radiation therapy is induced by the insertion of alpha-emitter seeds, the buffering time period (106) is selected to allow the dispersion of the alpha-emitting atoms in the tumor to a point at which the number of alpha-emitting radionuclides dispersed remote from the seeds is maximal.

The buffering time period (106) between implanting (104) the alpha emitter sources and the first session of administering (108) the vascular inhibitor is optionally at least 6 hours, at least 9 hours, at least 12 hours, at least 24 hours, at least 48 hours, at least 96 hours, at least 120 hours or even at least 144 hours. Optionally, the buffering time period (106) is shorter than two weeks, shorter than 10 days, shorter than a week, shorter than 120 hours, shorter than 96 hours, shorter than 72 hours or even shorter than 48 hours, so that the alpha emitting atoms leakage is minimized when the vascular inhibitor is applied. In some embodiments, the buffering time period (106) is shorter than 30 hours, shorter than 20 hours or even shorter than 10 hours, for example in tumors having large numbers of blood vessels or which otherwise react more quickly to the alpha-emitter radiotherapy.

In a third class of embodiments, the vascular inhibitor is administered after most of the radionuclides on the implanted seed underwent radioactive decay, which occurs within about two weeks. In this class of embodiments, the vascular inhibitor prevents the neovascularization of the tissue and/or the reestablishment of residual tumor cells from tumor cells that escaped the alpha-based therapy, in a wound healing reaction that the tissue damage following intensive tumor cell-killing, may invoke.

In some embodiments, after implantation and/or activation of alpha-emitter seeds, one or more parameters of the tumor are monitored in order to determine a time point most suitable for applying the vascular inhibitors therapy. The monitoring optionally includes imaging the tumor, using a suitable modality (e.g., X-ray, ultrasound, PET-CT, MRI, CT) to identify when the tumor begins to change due to the activation of alpha-emitter radiotherapy and/or to monitor a vascularization status of the tumor.

Alternatively, the monitoring includes performing a blood test to identify levels of an attribute. It is noted, however, that in some embodiments the vascular inhibitors therapy is applied before the effect of the alpha-emitter radiotherapy is detectable.

### Alpha-Emitter Radiation

The alpha radiation optionally includes insertion of seeds carrying alpha emitting atoms, such as Radium-224 or Radium-223, into the tumor. Optionally, the alpha emitting atoms are attached to the seed in a manner that the atoms do not leave the seed, but upon radionuclide decay resulting daughter radionuclides leave the seed. The brachytherapy seed optionally emits daughter radionuclide atoms at a rate of at least 0.1%, 0.5% or even at least 1% of the number of radionuclide atoms coupled to the seed when originally employed, per 24 hours. In some embodiments, the daughter radionuclide atoms are slowly released from the seed, at a rate of less than 25%, less than 10%, less than 5% or even less than 3% of the radionuclide atoms coupled to the seed, per 24 hours.

Alternatively to attaching the alpha emitting atoms to the seed in a manner that the atoms do not leave the seed without radionuclide decay, the alpha emitting atoms are attached to the seed in a manner that the atoms controllably leave the seed at a rate of at least 0.1% per 24 hours, in methods other than radionuclide decay, as described, for example in PCT publication WO 2019/193464, titled “controlled release of radionuclides”, which is incorporated herein by reference.

The diffusing alpha-emitters radiation therapy (DaRT) is optionally carried out using any of the methods and/or devices described in U.S. Pat. No. 8,834,837, US patent publication 2009/0136422, U.S. provisional application 62/913,184, filed Oct. 10, 2019, and/or PCT publication WO 2018/207105, which are incorporated herein by reference.

The alpha-emitter radiation treatment is optionally initiated by inserting (104) one or more brachytherapy seeds carrying alpha emitting atoms on an outer surface of the seeds, into the tumor. Alternatively, the alpha-emitter radiotherapy is initiated by activating previously inserted seeds, carrying alpha emitting atoms. Optionally, in accordance with this alternative, the seeds are inserted into the patient with a bio-absorbable coating, which prevents alpha-radiation and/or the daughter radionuclides from leaving the seeds. The bio-absorbable coating optionally comprises polylactide (PLA), polyglycolide (PGA) or co-polymers of PLA and PGA, tailored to achieve a desired resorption rate of the coating. Alternatively, or additionally, the coating comprises co-poly lactic acid/glycolic acid (PLGA). The polymers of the coating optionally have molecular weights ranging from 5,000 to 100,000. The material of the coating dissolves in the patient through any of the methods known in the art, such as one or more of ultrasonic energy, reaction with body temperature and/or reaction with body fluids. Additional discussion of bio-absorbable polymers which may be used in accordance with embodiments of the present invention after adjustment for the desired resorption rate are described in U.S. Pat. No. 8,821,364 and US patent publication 2002/0055667, which are incorporated herein by reference. In some embodiments, the initiation (108) includes applying a stimulus which dissolves the coating and thus allows the alpha radiation and/or the daughter radionuclides to leave the seed. In other embodiments, the initiation (108) is achieved by the dissolving of the coating due to contact with tissue of the tumor, without further required initiation.

The alpha-emitter radiotherapy is optionally applied to the patient for at least 24 hours, at least 5 days or even at least 10 days. In some embodiments, the radiotherapy seeds are removed from the patient after a designated treatment duration. For example, the seeds are optionally removed during surgery for removal of the tumor. Alternatively, the seed is not removed. In some embodiments, the seed comprises a biodegradable material.

### Treatment Kit

FIG. 2 is a schematic illustration of a kit 200 for treatment of a patient in accordance with the method of FIG. 1. Kit 200 comprises a sterile package 202 including one or more alpha-emitter radiotherapy seeds 204, for insertion into a tumor, and one or more doses 216 of a vascular inhibitor.

Optionally, the seeds 204 are provided within a vial or other casing 206 which prevents radiation from exiting the casing. In some embodiments, the casing is filled with a viscous liquid, such as glycerin, which prevents radioactive atoms from escaping the casing 206, such as described in PCT application PCT/IB2019/051834, titled “Radiotherapy Seeds and Applicators”, the disclosure of which is incorporated herein by reference. In some embodiments, kit 200 further includes a seed applicator 208, which is used to introduce seeds 204 into the patient, as described in PCT application PCT/IB2019/051834. Optionally, applicator 208 is provided preloaded with one or more seeds 204 therein. In accordance with this option, separate seeds 204 in casings 206 are supplied for cases in which more than the number of preloaded seeds is required. Alternatively, seeds 204 in casings 206 are not provided in kit 200 and only seeds within applicator 208 are included in the kit 200.

As shown, the doses 216 of the vascular inhibitors are provided preloaded in one or more needles 210. In other embodiments, the doses 216 are provided in one or more containers or vials 220 and the needles are provided separately within sterile package 202 or are not provided in kit 200, at all.

In some embodiments, kit 200 includes a plurality of separate compartments, separated by suitable insulation, for substances which require storage at different temperatures. For example, a first compartment may include dry ice which keeps the substances in the first compartment at about −20° C., while a second compartment includes ice which keeps the substances in the second compartment at about 4° C.

The radiotherapy seeds 204 optionally comprise a metallic or non-metallic support, which is configured for insertion into a body of a subject. The seeds 204 further comprise radionuclide atoms of e.g., radium-224, on an outer surface, as described, for example, in U.S. Pat. No. 8,894,969, which is incorporated herein by reference. The radionuclide atoms are generally coupled to the seed in a manner such that radionuclide atoms do not leave the support, but upon radioactive decay, their daughter radionuclides may leave the seed 204 due to recoil resulting from the decay. The percentage of daughter radionuclides that leave the support due to decay is referred to as the desorption probability. The coupling of radiotherapy atoms to the seed is achieved, in some embodiments, by heat treatment. Alternatively or additionally, a coating covers the seed and atoms, in a manner which prevents release of the radionuclide atoms, and/or regulates a rate of release of daughter radionuclides, upon radioactive decay. Daughter radionuclides may pass through the coating and out of the seed 204 due to recoil or the recoil may bring them into the coating, from which they leave by diffusion.

Seed 204 comprises, in some embodiments, a seed for complete implant within a tumor of a patient, and may have any suitable shape, such as a rod or plate. Alternatively to being fully implanted, seed 204 is only partially implanted within a patient and is part of a needle, a wire, a tip of an endoscope, a tip of a laparoscope, or any other suitable probe.

In some embodiments, seed 204 is cylindrical and has a length of at least 1 millimeter, at least 2 millimeters, or even at least 5 millimeters. Optionally, the seeds 204 have a length of between 5-60 mm (millimeters). Seed 204 optionally has a diameter of 0.7-1 mm, although in some cases, sources of larger or smaller diameters are used. Particularly, for treatment layouts of small spacings, seed 204 optionally has a diameter of less than 0.7 mm, less than 0.5 mm, less than 0.4 mm or even not more than 0.3 mm.

### Experiments

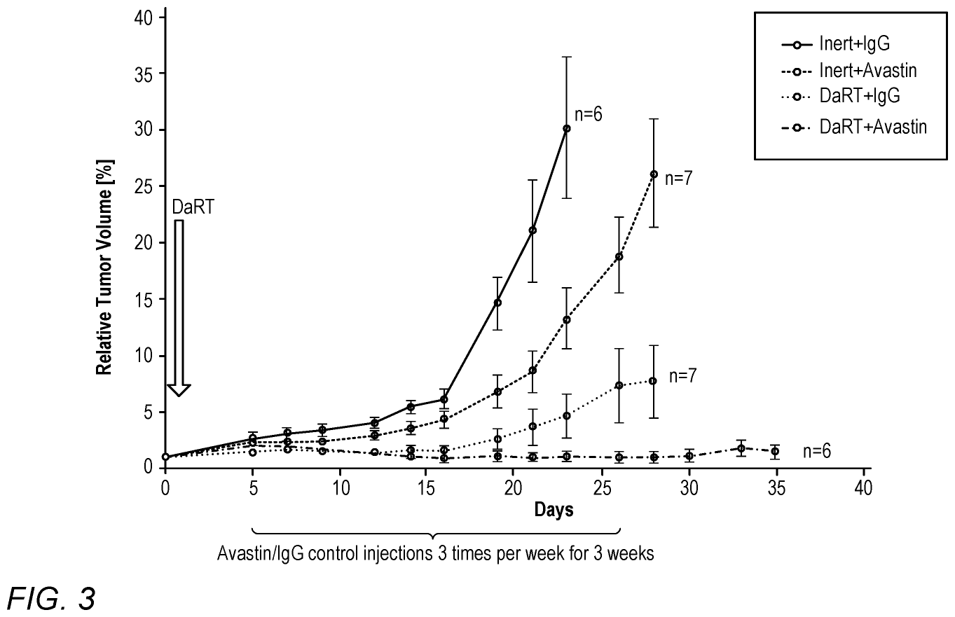

FIG. 3 shows the results of an experiment applicant performed to test the method of FIG. 1. In the experiment, the efficacy of DaRT with a systemic administration of BEV was tested on Glioblastoma Multiforme (GBM) xerographs. U87 cells were intracutaneously inoculated in a total of 5·10⁶ cells per mouse (in a volume of 100 μl) to the flank of athymic nude mice. The tumors were allowed to grow to an average size of 5-6 mm (longest diameter) for 9 days, when either a single DaRT or inert (non-radioactive control) seed was inserted to the middle of each tumor. Five days post seed insertion, each mouse received an i.p (intraperitoneal) administration of either IgG (control antibody) or BEV. This administration included 3 doses per week (5 mg/kg each) for 3 consecutive weeks (a total of 9 doses). The mice were divided to 4 treatment groups as follows:
a. Inert+Control IgG (untreated control);
b. Inert+BEV (Bev as a monotherapy);
c. DaRT+Control IgG (DaRT as a monotherapy);
d. DaRT+BEV (The combined modality)

Tumor volumes were monitored 3 times per week, along with radioactivity measurement using a Geiger counter to confirm that the DaRT seeds are present in the tumor.

As could be seen in FIG. 3, tumors in the Inert+IgG group grew rapidly and had to be eliminated after 23 days post treatment since they exceeded the allowed tumor volume. BEV as a standalone treatment provided a moderate but significant attenuation in tumor growth compared to the control (Inert+IgG) group. Furthermore, the DaRT-treated tumors were significantly smaller compared to the control as well as to the BEV-treated group and 2 out of 7 tumors were completely eradicated. Notably, a significant effect on tumor growth was observed in the combined therapy group compared to all other treatment groups. On the same note, 2 out of 6 tumors in the combination group were completely eradicated and declared as completely cured as they did not recur for a period of over 4 months. The experiment was repeated with similar results.

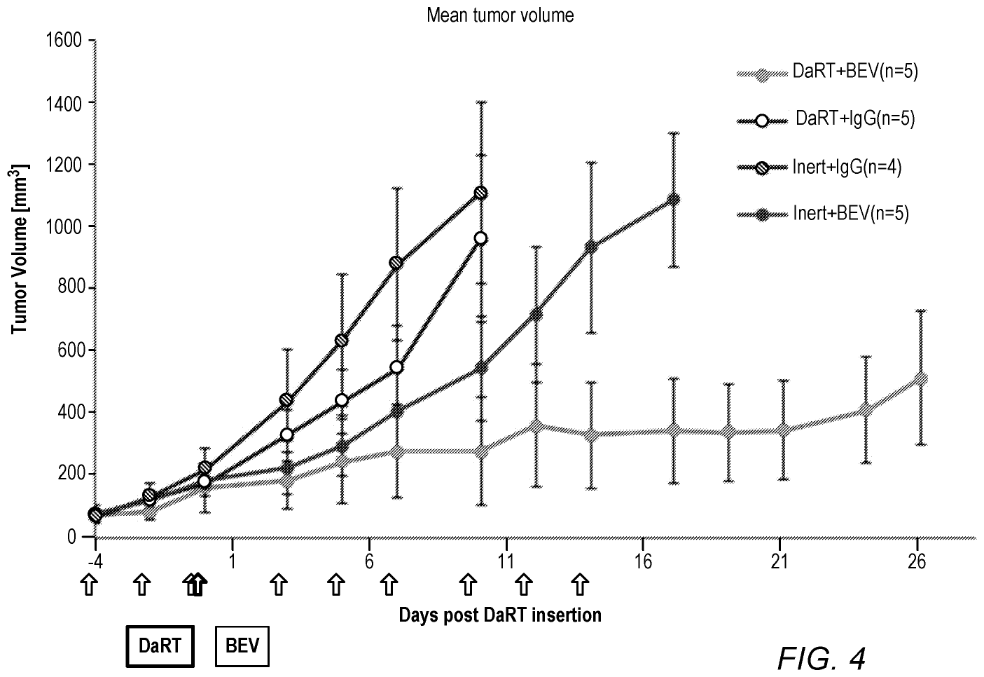

FIG. 4 shows the results of an experiment applicant performed to test the method of FIG. 1. In the experiment, the efficacy of DaRT with a systemic administration of BEV was tested in the same conditions as in FIG. 3, except for the timing of the BEV treatment-start, which was 4 days prior to DaRT insertion, and the tumor size at DaRT insertion was larger.

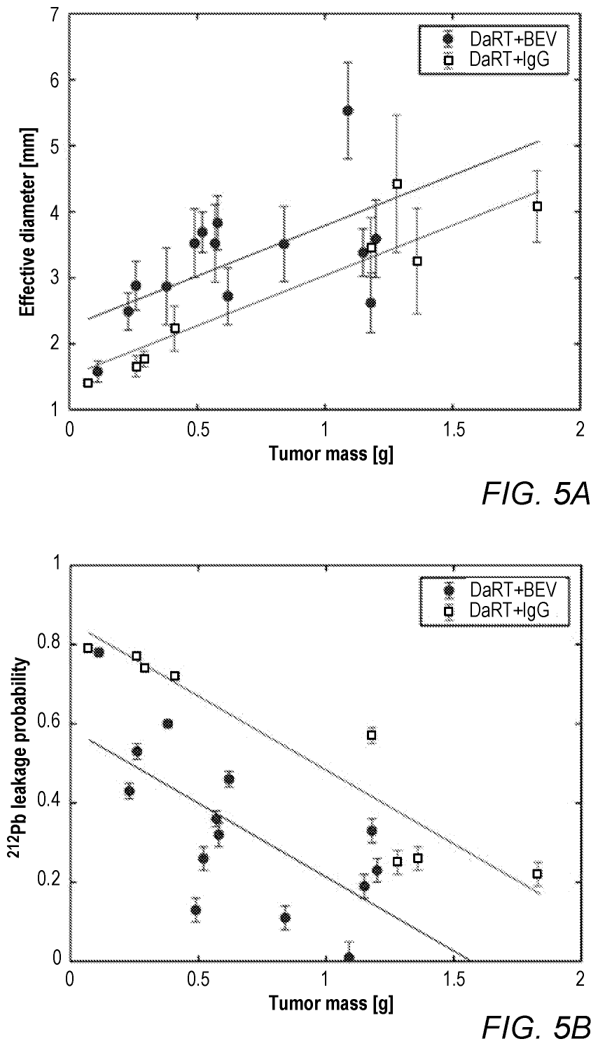

FIGS. 5A and 5B show the results of an experiment applicant performed to test the method of FIG. 1. In the experiment, autoradiography experiments were performed and the effective diameter (i.e., the distance from the DaRT seed for which the dose was greater than 10 Gy) of U87 tumors treated with DaRT and BEV was calculated and compared to the effective diameter of tumors treated with DaRT and IgG control (in the same treatment regimen mentioned in FIG. 4). Tumors were harvested 4-5 days post DaRT insertion. Analysis of covariance for effective diameter showed a significant difference between DaRT and BEV relative to DaRT and IgG in affecting the effective diameter (FIG. 5A) and the leakage (FIG. 5B) of radioactive atoms from the tumor to external organs, showing similar slopes but different intercepts. Effective diameter was significantly negatively correlated with the leakage from the tumor in both groups. This may suggest that BEV increased the effectiveness of DaRT by preventing the leakage of the radioactive atoms from the tumor thereby enhancing the effective diameter in the tumor.

### CONCLUSION

It will be appreciated that the above-described methods and apparatus are to be interpreted as including apparatus for carrying out the methods and methods of using the apparatus. It should be understood that features and/or steps described with respect to one embodiment may sometimes be used with other embodiments and that not all embodiments of the invention have all of the features and/or steps shown in a particular figure or described with respect to one of the specific embodiments. Tasks are not necessarily performed in the exact order described.

It is noted that some of the above-described embodiments may include structure, acts or details of structures and acts that may not be essential to the invention and which are described as examples. Structure and acts described herein are replaceable by equivalents which perform the same function, even if the structure or acts are different, as known in the art. The embodiments described above are cited by way of example, and the present invention is not limited to what has been particularly shown and described hereinabove. Rather, the scope of the present invention includes both combinations and sub-combinations of the various features described hereinabove, as well as variations and modifications thereof which would occur to persons skilled in the art upon reading the foregoing description and which are not disclosed in the prior art. Therefore, the scope of the invention is limited only by the elements and limitations as used in the claims, wherein the terms “comprise,” “include,” “have” and their conjugates, shall mean, when used in the claims, “including but not necessarily limited to.”

1. A vascular inhibitor for use as a medicament for treatment of a tumor of a patient wherein the administration pattern of the medicament comprises administering a therapeutically effective amount of the vascular inhibitor to the patient, in one or more sessions, and implanting seeds carrying radium-224 in the tumor for intra-tumoral alpha-emitter radiotherapy less than two weeks from administering the vascular inhibitor.

2. The vascular inhibitor as in claim 1, wherein the administration pattern of the medicament comprises beginning the administering of the vascular inhibitor within less than five days from implanting of the seeds.

3. The vascular inhibitor as in claim 1, wherein the administration pattern of the medicament comprises beginning the administering of the vascular inhibitor at least 12 hours after the implanting of the seeds.

4. The vascular inhibitor as in claim 1, wherein the administration pattern of the medicament comprises beginning the administering of the vascular inhibitor at least 72 hours after the implanting of the seeds.

5. The vascular inhibitor as in claim 1, wherein the administration pattern of the medicament comprises beginning the administering of the vascular inhibitor at least 12 hours before the implanting of the seeds.

6. The vascular inhibitor as in claim 1, wherein the administration pattern of the medicament comprises beginning the administering of the vascular inhibitor at least 72 hours before the implanting of the seeds.

7. The vascular inhibitor as in claim 1, wherein the vascular inhibitor comprises an antiangiogenic agent.

8. The vascular inhibitor as in claim 7, wherein the antiangiogenic agent comprises a vascular endothelial growth factor (VEGF) blockade.

9. The vascular inhibitor as in claim 8, wherein the VEGF blockade comprises an antibody against vascular endothelial growth factors (VEGFs).

10. The vascular inhibitor as in claim 9, wherein the antibody against VEGFs comprises Bevacizumab.

11. The vascular inhibitor as in claim 8, wherein the VEGF blockade comprises a vascular endothelial growth factor (VEGF) decoy receptor.

12. The vascular inhibitor as in claim 11, wherein the VEGF decoy receptor comprises Ziv-aflibercept.

13. The vascular inhibitor as in claim 8, wherein the VEGF blockade comprises an antibody against VEGF receptors.

14. The vascular inhibitor as in claim 7, wherein the antiangiogenic agent comprises a Kinase inhibitor.

15. The vascular inhibitor as in claim 14, wherein the Kinase inhibitor comprises at least one of Sorafenib, Sunitinib, Regorafenib and Lenvatinib.

16. The vascular inhibitor as in claim 7, wherein the antiangiogenic agent comprises an Immunomodulatory imide drug (IMiD).

17. The vascular inhibitor as in claim 16, wherein the Immunomodulatory imide drug (IMiD) comprises at least one of Thalidomide, Pomalidomide and lenalidomide.

18. The vascular inhibitor as in claim 7, wherein the antiangiogenic agent comprises an antiangiogenic microRNA.

19. The vascular inhibitor as in claim 7, wherein the antiangiogenic agent comprises an Endothelin receptor blocker.

20. The vascular inhibitor as in claim 7, wherein the antiangiogenic agent comprises Bosentan.

21. The vascular inhibitor as in claim 7, wherein the antiangiogenic agent comprises an mTOR inhibitor.

22. The vascular inhibitor as in claim 7, wherein the antiangiogenic agent comprises rapamycin.

23. The vascular inhibitor as in claim 7, wherein the antiangiogenic agent comprises a Fibroblast Growth Factors inhibitor.

24. The vascular inhibitor as in claim 7, wherein the antiangiogenic agent comprises Brivanib.

25. The vascular inhibitor as in claim 7, wherein the antiangiogenic agent comprises an Angiopoietins inhibitor.

26. The vascular inhibitor as in claim 7, wherein the antiangiogenic agent comprises a Platelet-Derived Growth Factor inhibitor.

27. The vascular inhibitor as in claim 7, wherein the antiangiogenic agent comprises ponatinib.

28. The vascular inhibitor as in claim 7, wherein the antiangiogenic agent comprises a Hepatocyte Growth Factor (HGF)/c-MET inhibitor.

29. The vascular inhibitor as in claim 7, wherein the antiangiogenic agent comprises a naturally anti-angiogenic factor or a derivative or a mimic of a naturally anti-angiogenic factor.

30. The vascular inhibitor as in claim 7, wherein the antiangiogenic agent comprises Endostatin.

31. The vascular inhibitor as in claim 8, wherein the antiangiogenic agent comprises one or more β-Adrenergic Drug.

32. The vascular inhibitor as in claim 8, wherein the antiangiogenic agent comprises Propranolol.

33. The vascular inhibitor as in claim 8, wherein the antiangiogenic agent comprises an angiogenesis inhibitor.

34. The vascular inhibitor as in claim 33, wherein the antiangiogenic agent comprises Metformin or Chloroquine.

35. The vascular inhibitor as in claim 8, wherein the antiangiogenic agent comprises a Cannabinoid.

36. The vascular inhibitor as in claim 8, wherein the antiangiogenic agent comprises a Matrix metalloproteinase inhibitor.

37. The vascular inhibitor as in claim 8, wherein the antiangiogenic agent comprises an inhibitor of integrin's pro-angiogenic activity.

38. The vascular inhibitor as in claim 1, wherein the vascular inhibitor comprises a vascular disrupting agent.

39. The vascular inhibitor as in claim 38, wherein the vascular disrupting agent comprises Combretastatin A-4 phosphate (CA4P).

40. The vascular inhibitor as in claim 1, wherein the seeds comprise:
a support having a length of at least 1 millimeter; and
radium-224 atoms coupled to the support such that not more than 20% of the radium-224 atoms leave the support into the tumor in 24 hours, without decay, when the seed is implanted in the tumor, but upon decay, at least 5% of daughter radionuclides of the radium-224 atoms leave the support upon decay.

41. A method of treating a patient with a tumor, comprising:
treating the tumor with intra-tumoral alpha-emitter radiotherapy; and
administering, to the patient, a vascular inhibitor within two weeks of beginning the treating of the tumor with intra-tumoral alpha-emitter radiotherapy.

42. The method of claim 41, wherein administering the vascular inhibitor comprises administering a vascular endothelial growth factor (VEGF) blockade.

43. The method of claim 41, wherein treating the tumor with intra-tumoral alpha-emitter radiotherapy comprises treating a colorectal cancer tumor.

44. The method of claim 43, wherein administering the vascular inhibitor comprises administering Bevacizumab.

45. The method of claim 41, wherein administering the vascular inhibitor comprises administering Ranibizumab.

46. The method of claim 41, wherein administering the vascular inhibitor comprises administering an antiangiogenic agent.

47. The method of claim 41, wherein administering the vascular inhibitor comprises administering a vascular endothelial growth factor (VEGF) inhibitor.

48. The method of claim 41, wherein administering the vascular inhibitor comprises administering a vascular disrupting agent.

49. The method of claim 41, wherein treating the tumor with intra-tumoral alpha-emitter radiotherapy comprises implanting a plurality of seeds each having a length of at least 1 millimeter, in the tumor.

50. The method of claim 49, wherein administering the vascular inhibitor comprises administering within less than five days from implanting of the seeds.

51. The method of claim 49, wherein administering the vascular inhibitor comprises administering at least 12 hours after the implanting of the seeds.

52. An alpha-emitting device designed for use in intra-tumoral alpha-emitter radiotherapy treatment of a tumor of a patient, wherein the alpha-emitter radiotherapy treatment pattern comprises implanting the intra-tumoral alpha-emitter device in the tumor and administering a therapeutically effective amount of a vascular inhibitor, in one or more sessions, within two weeks before or after beginning the alpha-emitter radiotherapy.

53. The device as in claim 52, wherein the device comprises:
a support having a length of at least 1 millimeter; and
radium-224 atoms coupled to the support such that not more than 20% of the radium-224 atoms leave the support into the tumor in 24 hours, without decay, when the device is implanted in the tumor, but upon decay, at least 5% of daughter radionuclides of the radium-224 atoms leave the support upon decay.
